(19) United States
(12) Patent Application Publication
(10) Pub. No.: US 2024/0315599 A1
(43) Pub. Date: Sep. 26, 2024

(54) BONE CONDUCTION-BASED EATING MONITORING METHOD AND APPARATUS, TERMINAL DEVICE, AND MEDIUM

(71) Applicant: GOERTEK INC., Weifang, Shandong (CN)

(72) Inventors: Xiaochen WANG, Weifang, Shandong (CN); Xiaoyu GE, Weifang, Shandong (CN)

(21) Appl. No.: 18/578,871

(22) PCT Filed: Dec. 16, 2021
(86) PCT No.: PCT/CN2021/138685

§ 371 Date: Jan. 12, 2024

(30) Foreign Application Priority Data
Jul. 22, 2021 (CN) 202110833917.X

(51) Int. Cl. A61B 5/11; A61B 5/00

(52) U.S. Cl. CPC A61B 5/1114; A61B 5/1126; A61B 5/6803

(57) ABSTRACT

Provided are a bone conduction-based eating monitoring method and apparatus, a terminal device, and a storage medium, the method including monitoring, by means of smart glasses, a chewing behavior for food being eaten to obtain a vibration signal of skull related to the chewing behavior; counting the number of chews according to the vibration signal to obtain the number of consecutive chews; and if the number of consecutive chews does not reach a target number of chews and the chewing behavior of a user terminates, sending first prompt information; if the number of consecutive chews reaches the target number of chews, sending second prompt information.

### CROSS REFERENCE TO RELATED APPLICATION

This application claims priority to Chinese Patent Application No. 202110833917.X, entitled “bone conduction-based eating monitoring method and apparatus, terminal device, and medium”, filed with China Patent Office on Jul. 22, 2021, which is hereby incorporated by reference as if fully set forth herein.

### TECHNICAL FIELD

The present disclosure relates to a technical field of intelligent monitoring, and more particularly, to a bone conduction-based eating monitoring method and apparatus, terminal device, and storage medium.

### BACKGROUND

with the consecutive improvement of living standards, people's demand for a healthy diet is also increasing. Among them, the chewing habit during eating is closely related to a healthy diet, and therefore have received people's special attention. Scientific research has shown that the more times people chew, the more calory people consume. Since satiety has the delayed feeling, in the case of eating the same amount of food, chewing more times may lead to more effective weight loss.

If the number of chews is calculated subjectively, it may exert a burden on people's diet and is also not conducive to improving intervention measures aimed at changing chewing rates. There are currently relevant studies on methods for monitoring the chewing process, but all of them have shortcomings. For example, the method of monitoring chewing behavior by monitoring chewing sound may have the problem that environmental noise interferes with the monitoring process, thereby reducing the accuracy of monitoring results. The method of monitoring chewing behavior through smart glasses includes monitoring temporal muscle activity by using piezoelectric thin film sensors and recording temporal muscles by using electromyography. The former is limited to the experimental stage and involves signal segmentation which may result in insufficient accuracy; the latter may also affect the accuracy of monitoring results since the operations such as filtering and removing motion artifacts are needed due to the excessive noise signal.

Therefore, there is currently no objective and automated method for quantifying chewing behavior that can automatically monitor the number of chews with a more reliable accuracy and thus improve the eating habit of the user.

### SUMMARY

The main purpose of the present application is to provide a bone conduction-based eating monitoring method and apparatus, terminal device, and storage medium, aiming to automatically monitor the number of chews with more reliable accuracy, and timely send prompt information to a user based on user's chewing behavior, thereby improving his eating habit.

In order to achieve the above objective, the present application provides a bone conduction-based eating monitoring method, which is applied to wearable device. The bone conduction-based eating monitoring method includes:
monitoring a chewing behavior for food being eaten to obtain a vibration signal of skull related to the chewing behavior;
counting the number of chews according to the vibration signal to obtain the number of consecutive chews; and
if the number of consecutive chews does not reach a target number of chews and the chewing behavior of a user terminates, sending a first prompt information; and
if the number of consecutive chews reaches the target number of chews, sending a second prompt information.

Furthermore, after counting the number of chews according to the vibration signal to obtain the number of consecutive chews, the bone conduction-based eating monitoring method includes:
determining whether the number of consecutive chews reaches a preset monitoring value; and
if the number of consecutive chews reaches the preset monitoring value, obtaining the target number of chews.

Furthermore, after determining whether the number of consecutive chews reaches a preset monitoring value, the bone conduction-based eating monitoring method further includes:

If the number of consecutive chews reaches the preset monitoring value, activating an eating monitoring function of the wearable device and sending a prompt message to the user.

Furthermore, before obtaining the target number of chews, the bone conduction-based eating monitoring method further includes:
obtaining a preset instruction; and
determining the target number of chews according to the preset instruction.

Furthermore, before obtaining target number of chews, the bone conduction-based eating monitoring method further includes: obtaining the type of food being eaten; and
determining the target number of chews according to the type of food.

Furthermore, after obtaining a vibration signal of skull related to the chewing behavior, the bone conduction-based eating monitoring method further includes:

counting a vibration interval according to the vibration signal.

After counting the number of chews according to the vibration signal to obtain the number of consecutive chews, the bone conduction-based eating monitoring method further includes:
if the vibration interval reaches a preset duration period, resetting the number of consecutive chews; and
counting the number of chews according to real-time vibration signal and updating the number of consecutive chews.

Furthermore, before monitoring a chewing behavior for food being eaten to obtain a vibration signal of skull related to the chewing behavior, the bone conduction-based eating monitoring method further includes:
obtaining a monitoring instruction; and
activating an eating monitoring function according to the monitoring instruction.

In addition, in order to achieve the above purpose, the present application further provides a bone conduction-based eating monitoring apparatus, wherein the bone conduction-based eating monitoring apparatus is applied to wearable device, and the bone conduction-based eating monitoring apparatus includes:
a monitoring module for monitoring a chewing behavior for food being eaten to obtain a vibration signal of skull related to the chewing behavior;
a counting module for counting the number of chews according to the vibration signal to obtain the number of consecutive chews;
a prompting module for sending a first prompt message if the number of consecutive chews does not reach the target number of chews and the chewing behavior of a user terminates, and sending a second prompt message if the number of consecutive chews reaches the target number of chews.

Each of the functional modules of the bone conduction-based eating monitoring apparatus according to the present application implement the steps of the bone conduction-based eating monitoring method as described above during operation.

Furthermore, in order to achieve the above purpose, the present application further provides a terminal device, including a memory, a processor, and bone conduction-based eating monitoring program stored on the memory and operable on the processor. The steps of the bone conduction-based eating monitoring method as described above are implemented when the bone conduction-based eating monitoring program is executed by the processor.

In addition, in order to achieve the above purpose, the present application further provides a storage medium on which a computer program is stored, the steps of the bone conduction-based eating monitoring method as described above are implemented when the computer program is executed by the processor.

In addition, the present embodiment further proposes a computer program product, which includes a bone conduction-based eating monitoring program. The steps of implementing the bone conduction-based eating monitoring method as described above are implemented when the bone conduction-based eating monitoring program is executed by the processor.

Here, the steps implemented when the bone conduction-based eating monitoring program running on the processor is executed may refer to various embodiments of the bone conduction-based eating monitoring method according to the present application, and will not be repeated herein.

The present application proposes a bone conduction-based eating monitoring method and apparatus, terminal device, and storage medium, including monitoring the chewing behavior during eating through wearable device to obtain a vibration signal of skull related to the chewing behavior, counting the number of chews according to the vibration signal to obtain the number of consecutive chews, and sending a first prompt information if the number of consecutive chews does not reach a target number of chews and the chewing behavior of a user terminates and sending a second prompt information if the number of consecutive chews reaches the target number of chews.

The present application monitors, by a wearable device, a user's chewing behavior of food being eaten when the user is eating food to obtain a vibration signal of skull related to the chewing behavior, counts the vibration number of the skull according to the vibration signal, determines the user's number of chews according to the vibration number of the skull, and obtains the number of consecutive chews according to the number of chews. The number of consecutive chews is equivalent to the number of consecutive chews of the user's chewing behavior. Finally, the wearable device compares the number of consecutive chews with the target number of chews, and sends a first prompt information if the number of consecutive chews does not reach the target number of chews and the chewing behavior of a user terminates, and the wearable device sends a second prompt information if the number of consecutive chews reaches the target number of chews.

The present application monitors the vibration number of skull related to the user's chewing behavior by the wearable device, to achieve the monitoring for the eating habit of the user, and collects the vibration signal of the skull related to the chewing behavior according to bone conduction technology. The vibration signal of the skull is simple and reliable. Meanwhile, collecting the vibration signal of skull does not involve oral recordings. Thus, the monitoring process is not sensitive to the noise of the surrounding environment and has strong anti-interference ability and a higher accuracy of monitoring results. In addition, when the wearable device determines that the number of consecutive chews reaches the target number of chews or the number of consecutive chews does not reach the target number of chews and the chewing behavior of a user terminates during the chewing behavior, the wearable device will send a prompt message to the user to serve as reminders, thereby achieving the purpose of improving the eating habit of the user.

In this way, the bone conduction-based eating monitoring method provided in the present application can automatically monitor the number of chews with more reliable accuracy, and timely send prompt information to users according to the user's chewing behavior, thereby improving the eating habit of the user.

### BRIEF DESCRIPTION OF DRAWINGS

FIG. 1 is a structural schematic diagram of the hardware operating environment of the terminal device according to the embodiment of the present application;

FIG. 2 is a flowchart schematic diagram of an embodiment of the bone conduction-based eating monitoring method according to the present application;

FIG. 3 is a detailed flowchart schematic diagram of an embodiment of the bone conduction-based eating monitoring method according to the present application;

FIG. 4 is a schematic diagram of the skull involved in human biting behavior;

FIG. 5 is a schematic diagram illustrating a case in which the user wears smart glasses to monitor the number of chews in an embodiment of the bone conduction-based eating monitoring method according to the present application;

FIG. 6 is a schematic diagram of the module structure of the bone conduction-based eating monitoring apparatus according to the present application.

The implementation, functional characteristics, and advantages of the purpose of the present application will be further explained in conjunction with the embodiments, with reference to the accompanying drawings.

### DETAILED DESCRIPTION

It should be understood that the specific embodiments described herein are only intended to explain the present application and are not intended to limit the present application.

As shown in FIG. 1, FIG. 1 is a structural schematic diagram of the hardware operating environment of the terminal device according to the embodiment of the present application.

It should be noted that FIG. 1 is a structural schematic diagram of the hardware operating environment of the terminal device. The terminal device of the embodiment of the present application may be smart glasses.

As shown in FIG. 1, the terminal device may include: a processor 1001, such as CPU, a network interface 1004, a user interface 1003, a memory 1005, and a communication bus 1002. Among them, the communication bus 1002 is used to achieve connection communication between these components. User interface 1003 may include a display screen (Display), an input unit such as a keyboard, and optional user interface 1003 may also include standard wired interface and wireless interface. The network interface 1004 may optionally comprise a standard wired interface and wireless interface (such as WI-FI interfaces). Memory 1005 may be a high-speed RAM memory, and may also be a stable memory (non-volatile memory), such as disk storage. Memory 1005 may optionally be a storage device independent of the aforementioned processor 1001.

Those skilled in the art can understand that the structure of the terminal device shown in FIG. 1 is not a limitation to the terminal device, and may comprise more or fewer components than that shown in the illustration, or combinations of certain components, or different component arrangements.

As shown in FIG. 1, the memory 1005 as a computer storage medium may include an operating system, a network communication module, a user interface module, and a distributed task processing program. Among them, the operating system is a program that manages and controls the hardware and software resources of sample terminal devices, and supports distributed task processing programs, and other software or program operations.

In the terminal device shown in FIG. 1, the user interface 1003 is mainly used for data communication with various terminals; the network interface 1004 is mainly used to connect to the backend server and communicate data with the backend server; and the processor 1001 can be used to call the bone conduction-based eating monitoring program stored in memory 1005 and further perform the following operations:
monitoring a chewing behavior for food being eaten to obtain a vibration signal of skull related to the chewing behavior;
counting the number of chews according to the vibration signal to obtain the number of consecutive chews;
sending a first prompt information if the number of consecutive chews does not reach a target number of chews and the chewing behavior of a user terminates;
sending a first prompt information if the number of consecutive chews does not reach a target number of chews and the chewing behavior of a user terminates; and sending a second prompt information if the number of consecutive chews reaches the target number of chews.

Furthermore, processor 1001 can call the bone conduction-based eating monitoring program stored in the memory 1005, and further perform the following operations after the step of counting the number of chews according to the vibration signal to obtain the number of consecutive chews. The operations include:
determining whether the number of consecutive chews reaches the preset monitoring value; and
if the number of consecutive chews reaches the preset monitoring value, obtaining the target number of chews.

Furthermore, the processor 1001 can call the bone conduction-based eating monitoring program stored in memory 1005, and further perform the following operation after the step of determining whether the number of consecutive chews reaches the preset monitoring value. The operation includes:

If the number of consecutive chews reaches the preset monitoring value, activating an eating monitoring function of the wearable device and sending a prompt message to the user.

Furthermore, the processor 1001 can call the bone conduction-based eating monitoring program stored in the memory 1005, and further perform the following operations before the step of obtaining the target number of chews. The operations include:
obtaining a preset instruction; and
determining the target number of chews according to the preset instruction.

Furthermore, the processor 1001 can call the bone conduction-based eating monitoring program stored in the memory 1005, and further perform the following operations before the step of obtaining target number of chews. The operations include:
obtaining the type of food being eaten; and
determining the target number of chews according to the type of food.

Furthermore, the processor 1001 can call the bone conduction-based eating monitoring program stored in the memory 1005, and further perform the following operation after the step of obtaining a vibration signal of skull related to the chewing behavior. The operation includes:
counting a vibration interval according to the vibration signal.

The processor 1001 can call the bone conduction-based eating monitoring program stored in the memory 1005, and further perform the following operations after the step of counting a vibration interval according to the vibration signal. The operations include:
if the vibration interval reaches a preset duration period, resetting the number of consecutive chews; and
counting the number of chews according to real-time vibration signal and updating the number of consecutive chews.

The processor 1001 can call the bone conduction-based eating monitoring program stored in the memory 1005, and further perform the following operations before the step of monitoring a chewing behavior for food being eaten to obtain a vibration signal of skull related to the chewing behavior. The operations include:
obtaining a monitoring instruction; and
activating an eating monitoring function according to the monitoring instruction.

Various embodiments of the bone conduction-based eating monitoring method according to the present application are proposed based on the above structure.

It should be noted that there is currently relevant studies on methods for monitoring the chewing process, but all of them have shortcomings. For example, the method of monitoring chewing behavior by monitoring chewing sound may have the problem that environmental noise interferes with the monitoring process and thereby reducing the accuracy of monitoring results. The method of monitoring chewing behavior through smart glasses includes monitoring temporal muscle activity by using piezoelectric thin film sensors and recording temporal muscles by using electromyography. The former is limited to the experimental stage and involves signal segmentation which may result in insufficient accuracy; the latter may also affect the accuracy of monitoring results since the operations such as filtering and removing motion artifacts are needed due to the excessive noise signal.

Therefore, there is currently no objective and automated method for quantifying chewing behavior that can automatically monitor the number of chews with more reliable accuracy and thereby improving the eating habit of the user.

Based on the above situation, various embodiments of the bone conduction-based eating monitoring method according to the present application are proposed It should be noted that although the logical order is shown in the flowchart, in some cases, the steps shown or described can be executed in an order different from that described herein.

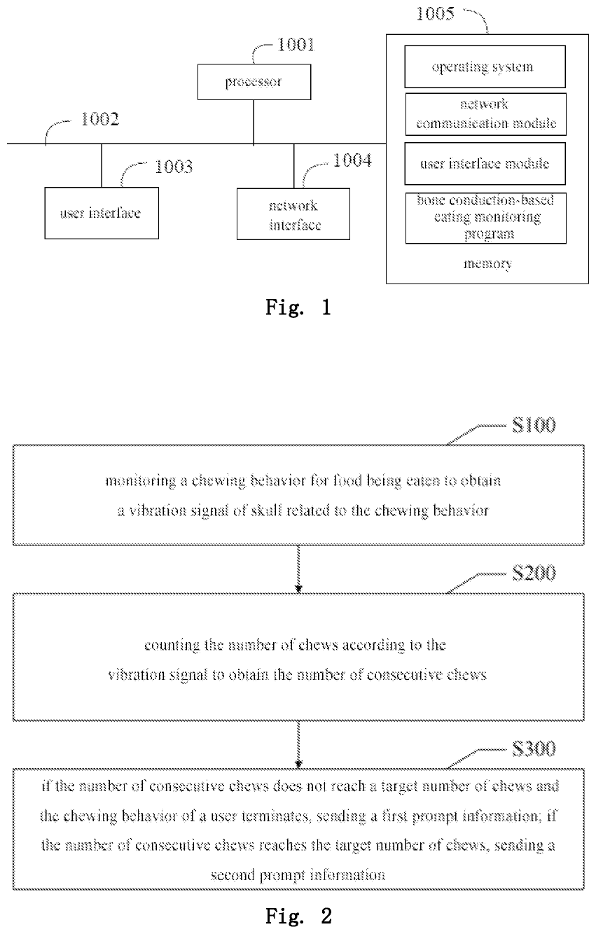

First embodiment: referring to FIG. 2, which is a flowchart schematic diagram of an embodiment of the bone conduction-based eating monitoring method according to the present application. The bone conduction-based eating monitoring method provided in the present application is applied to wearable device, and the bone conduction-based eating monitoring method according to the present application includes:

Step S100, monitoring a chewing behavior for food being eaten to obtain a vibration signal of skull related to the chewing behavior.

It should be noted that in this embodiment, the bone conduction-based eating monitoring method is applied to smart glasses, therefore the process of monitoring the number of chews is mainly completed through monitoring by smart glasses.

Smart glasses monitor the user's chewing behavior during eating stage to obtain a vibration signal of skull related to the chewing behavior.

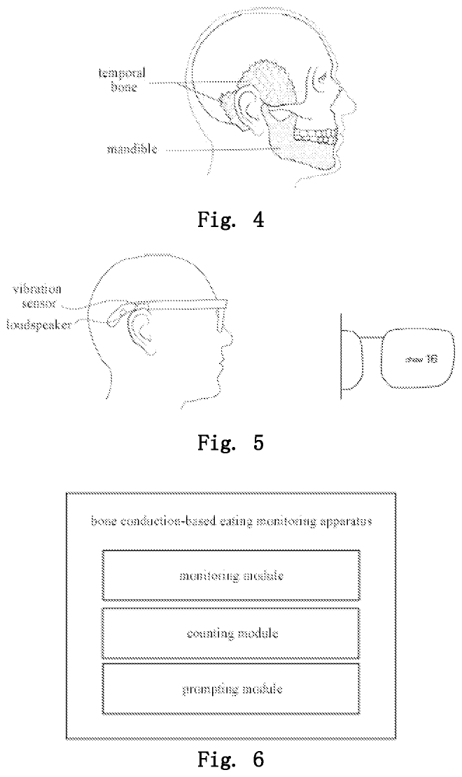

Specifically, as shown in FIGS. 4 and 5, the human chewing system is composed of teeth, temporomandibular joints, and jawbones. The chewing movement is mainly caused by the contraction and relaxation of the chewing muscles, thereby driving the mandible to achieve jaw closing and opening movements. When a person is chewing, the biting motion may drive the temporal bone of the head to vibrate, and when a person wears smart glasses, the legs of the smart glasses directly contact the head. Each biting motion of the person can be easily obtained by the bone conduction microphone installed on the legs of the glasses. Therefore, when a user is chewing during eating, the biting motion of the jawbone may drive the temporal bone of the head to vibrate, and the bone conduction microphone installed on the smart glasses obtains the vibration signal of the temporal bone, and monitors chewing behavior by obtaining this vibration signal.

The bone conduction-based eating monitoring method according to the present application includes: Step S200, counting the number of chews according to the vibration signal to obtain the number of consecutive chews.

After obtaining a vibration signal of skull related to the user's chewing behavior during eating, the smart glasses count the vibration number of the skull according to the vibration signal, and count the number of chews by the user according to the vibration number of the skull, so as to obtain the number of consecutive chews by the user over a period of time.

Specifically, for example, after obtaining the vibration signal of the user's temporal bone during the eating stage by the bone conduction microphone installed on the legs of the smart glasses, the smart glasses transmit the vibration signal to the vibration sensor installed on the smart glasses, and count the vibration number of the temporal bone according to the vibration signal, and then count the number of chews by the user according to the vibration number of the skull, to obtain the number of consecutive chews by the user over a period of time.

The number of consecutive chews is equivalent to the number of consecutive chews of the user's chewing behavior. Therefore, for each chew by the user, the vibration sensor obtains a vibration signal and accumulates the number of chews, thereby achieving monitoring of the number of consecutive chews by the user during the eating stage.

The bone conduction-based eating monitoring method according to the present application includes: Step S300, if the number of consecutive chews does not reach a target number of chews and the chewing behavior of a user terminates, sending a first prompt information; and if the number of consecutive chews reaches the target number of chews, sending a second prompt information.

It should be noted that, in this embodiment, the target number of chews is a measurement standard used to measure whether the number of chews by the user during the eating period reaches the target value; if the number of consecutive chews does not reach a target number of chews and the chewing behavior of a user terminates, a first prompt information is sent. The user's chewing behavior is determined as terminated by not performing chewing behavior any more within the preset time interval since the last chewing behavior. At this time, it can be understood as the user having finished the chewing behavior and having completed swallow. The first prompt message may be sent to remind the user that the chewing behavior does not reach the target number of chews; if the number of consecutive chews reaches the target number of chews, a second prompt information is sent, and the second prompt message is used to remind the user that the chewing behavior reaches the target value of the number of chews. The expression forms of the first prompt information and the second prompt information include image form, sound form, or vibration form, and are not specifically restricted herein.

After obtaining the number of consecutive chews, the smart glasses compare the number of consecutive chews with the target number of chews. If the number of consecutive chews does not reach the target number of chews and the chewing behavior of a user terminates, the smart glasses send the first prompt message to the user in the form of image, the form of sound, or the form of vibration. If the number of consecutive chews reaches the target number of chews, the smart glasses send the second prompt message to the user in the form of image, the form of sound, or the form of vibration.

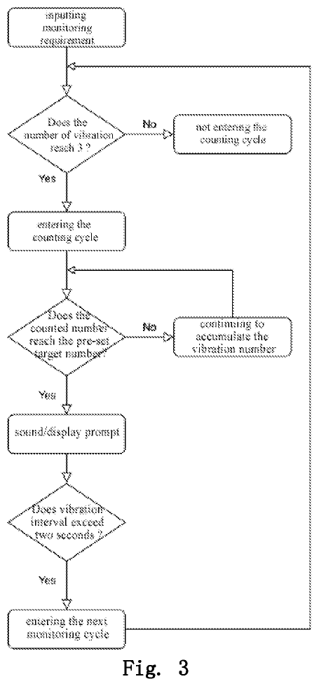

Specifically, as shown in FIG. 3, assuming that the target number of chews is 30 and the smart glasses monitor that the vibration number of the temporal bone reaches 30, the number of consecutive chews is 30, that is, at this point, the number of consecutive chews reaches the target number of chews. In this case, the smart glasses send the second prompt message. For example, the smart glasses display a second specific image by a display device, or emit a warning sound “beep, beep . . . ” by a sound output device, or generate two consecutive vibrations by specific parts of the smart glasses, thereby reminding users that the number of chews of the chewing behavior reaches the target value. On the contrary, assuming that the target number of chews is 30 and the smart glasses monitors that the vibration of the temporal bone terminates when vibration number only reaches 25, the number of consecutive chews is 25, that is, the chewing behavior is terminated and swallowing is completed before the number of consecutive chews reaches the target number of chews. In this case, the smart glasses send the first prompt message. For example, the smart glasses display the first specific image by a display device, or emit a warning sound “bee--------p” by a sound output device, or generate two consecutive vibrations by specific parts of the smart glasses, and reset the number of consecutive chews to 0.

In this embodiment, the user's chewing behavior during eating stage is monitored by the smart glasses to obtain a vibration signal of skull related to the chewing behavior; after obtaining the vibration signal of skull related to the user's chewing behavior during eating, the vibration number of the skull is counted according to the vibration signal, and then the user's chewing number is counted according to the vibration number of the skull, thereby obtaining the number of consecutive chews used to count the user's numbers of chewing; after obtaining the number of consecutive chews, the number of consecutive chews is compared with the target number of chews, and if the number of consecutive chews does not reach the target number of chews and the chewing behavior of a user terminates, a first prompt message is sent; if the number of consecutive chews reaches the target numbers of chewing, a second prompt message is sent.

The present application monitors the vibration number of skull related to the user's chewing behavior by the wearable device, thereby achieving monitoring of the eating habit of the user, and collects the vibration signal of the skull related to chewing behavior based on the bone conduction technology. The vibration signal is simple and reliable. Meanwhile, collecting the vibration signal of skull does not involve oral recordings. Thus, the monitoring process is not sensitive to the noise of the surrounding environment and has strong anti-interference ability and a higher accuracy of monitoring results. In addition, when the wearable device determines that the number of consecutive chews of the user in this chewing behavior does not reach the target number of chews and the chewing behavior of the user terminates, the wearable device sends a first prompt message. When the number of consecutive chews of the user in this chewing behavior reaches the target number of chews, the wearable device sends a second prompt message to the user, thereby achieving the purpose of improving the eating habit of the user.

Meanwhile, bone conduction technology is used to monitor the number of chews, and the data obtained during the monitoring process is vibration signal, and it is not necessary to adopt sound recording method. Therefore, it does not involve user's privacy issues.

In this way, the bone conduction-based eating monitoring method provided in the present application can automatically monitor the number of chews with more reliable accuracy, and timely send prompt information to users according to the user's chewing behavior, thereby improving the eating habit of the user.

Furthermore, in one embodiment, after step S200 described above, the bone conduction-based eating monitoring method may include:

Step S400, determining whether the number of consecutive chews reaches the preset monitoring value.

It should be noted that, in this embodiment, the preset monitoring value is a custom value pre-set by the user or manufacturer to avoid false triggering of the monitoring function of the smart glasses by the user's chewing behavior during the non-eating stage. The preset monitoring value may be 3, 4, or 5, etc., and it is not specifically limited thereto.

The smart glasses compare the number of consecutive chews with the custom value pre-set by the user or manufacturer.

The bone conduction-based eating monitoring method further includes: Step S500, if the number of consecutive chews reaches the preset monitoring value, obtaining the target number of chews.

After comparing the number of consecutive chews with the preset monitoring value, the eating monitoring function of the smart glasses enters the monitoring cycle and obtains the target number of chews for the user's chewing behavior if the number of consecutive chews reaches the preset monitoring value.

Specifically, for example, as shown in FIG. 3, it is assumed that the user sets the preset monitoring value to 3, and the smart glasses monitor the vibration signal of the user's temporal bone, that is, at this point, the user's chewing behavior is detected. If the vibration number of the user's temporal bone reaches 3, that is, at this point, the number of consecutive chews is 3, which reaches the preset monitoring value, then the smart glasses determine that the user is in the eating stage, and the eating monitoring function of the smart glasses enters the monitoring cycle and the smart glasses generate the target number of chews for the user's chewing behavior. Similarly, it is assumed that the user sets the preset monitoring value to 3 times and the smart glasses monitor the vibration signal of the user's temporal bone, that is, at this point, the user's chewing behavior is detected. If the vibration of the user's temporal bone terminates after the vibration number only reaches 2, that is, the number of consecutive chews is 2 and does not reach the preset monitoring value, then the smart glasses determine that the user is in the non-eating stage, and the monitoring function of the smart glasses does not enter the monitoring cycle, and the smart glasses reset the number of consecutive chews to 0.

In this way, in this embodiment, by setting preset monitoring value, the monitoring function of the smart glasses enters the monitoring cycle and obtains the target number of chews of the user's chewing behavior only when the number of consecutive chews of the user's chewing behavior reaches the preset monitoring value, thereby avoiding the false triggering of the monitoring function of the smart glasses by the user's chewing behavior during non-eating stage such as chewing gum, which may cause unnecessary interference to the user, and also reducing the power consumption of smart glasses.

Furthermore, in one embodiment, after the step of determining whether the number of consecutive chews reaches the preset monitoring value in step S500 described above, the method further includes:

Step S501, if the number of consecutive chews reaches the preset monitoring value, activating an eating monitoring function of the wearable device and sending a prompt message to the user.

After comparing the number of consecutive chews with the preset monitoring value, if the number of consecutive chews reaches the preset monitoring value, the smart glasses activate monitoring function of the wearable device and send a prompt message to the user. At this time, the monitoring function of the smart glasses enters the monitoring cycle and the smart glasses send a prompt message to the user. The prompt message may be in form of sound, vibration feedback, or image, etc. In this embodiment, image information reflecting the number of consecutive chews may be generated and displayed to the user by the display device.

Specifically, for example, it is assumed that the user sets the preset monitoring value to 3 and the smart glasses monitor the vibration signal of the user's temporal bone, that is, at this point, the user's chewing behavior is detected. If the vibration number of the user's temporal bone reaches 3, that is, at this point, the number of consecutive chews is 3, which reaches the preset monitoring value, then the smart glasses determine that the user is in the eating stage, and the eating monitoring function of the smart glasses enters the monitoring cycle, and the smart glasses send a prompt message to the user, such as, generating image information reflecting the number of consecutive chews and displaying the image information by the display device installed on the lens of the smart glasses to remind the user that the current number of consecutive chews is 3. As the user's chewing behavior continues, the image information is consecutively updated to reflect the number of consecutive chews of the user in real time.

In this way, this embodiment achieves synchronous feed back of the smart glasses to the user while monitoring the user's chewing behavior to remind the user to pay attention to the difference between the number of consecutive chews and the target number of chews, thereby improving the probability that the user achieves the target number of chews during the eating stage, and sending a prompt message to the user when the user completes chewing and swallowing before the number of consecutive chews reaches the target number of chews.

Furthermore, in one embodiment, before the step of obtaining the target number of chews in step S500 described above, the method further includes:

Step S502, obtaining the preset instruction.

It should be noted that, in this embodiment, the preset instruction is the instruction that the user autonomously triggers for the smart glasses, and is used to determine the target number of chews.

The smart glasses obtain an instruction that the user autonomously triggers to determine the target number of chews.

The method further includes: Step S503, determining the target number of chews according to the preset instruction.

The smart glasses determine the measurement standard used to measure whether the number of chews of the user during the eating period reaches the target value, according to the instruction autonomously triggered by the user.

Specifically, for example, the user inputs an instruction by the shortcut key of the smart glasses, autonomously sets the target number of chews to 30, or triggers the step of obtaining the target number of chews by a preset instruction. When receiving the preset instruction, the smart glasses obtain the corresponding target number of chews from the pre-stored correspondence table according to the type of food which the user is currently chewing. The pre-stored correspondence table can be pre-stored in the memory of the smart glasses, or may be obtained by the smart glasses from a cloud space through network. When entering the monitoring cycle, the monitoring function of smart glasses determines the number of 30 as a measurement standard used to measure whether the accumulated number of chews by the user during the eating stage reaches the target value, according to this instruction.

In this way, this embodiment enables the user to send the preset instruction to autonomously obtain the target number of chews, and therefore, the function of monitoring the user's eating by the smart glasses can meet the personalized needs and improve the user's using experience.

Furthermore, in another embodiment, before the step of obtaining the target number of chews in step S500 described above, the method further includes:

Step S504, obtaining the type of food being eaten.

After the monitoring function enters the food monitoring cycle, the smart glasses capture photos of the food being eaten by the user to obtain food images, and analyze the food images to obtain the type of food being eaten by the user.

Specifically, for example, assuming that the food being eaten by the user is beef, the smart glasses capture an image of the food being eaten by the user by a camera after the eating monitoring function enters the monitoring cycle, and then analyze the image to determine that the food belongs to the food type of “beef-meat-high calorie”.

The method further includes: Step S505, determining the target number of chews according to the type of food.

After determining the type of food, smart glasses determine the measurement standard used to measure whether the accumulated number of chews of the user's chewing behavior reaches the target value according to the food type.

Specifically, for example, assuming that the smart glasses capture photos of the food being eaten by the user through a camera and determine that the food belongs to the food type of “beef-meat-high calorie” after analyzing the food image, the smart glasses automatically determine the target number of chews as 30 according to the food type of “beef-meat-high calorie”, that is, the number of chews “30” is the target value that the user should achieve for this chewing behavior. furthermore, for example, assuming that the smart glasses capture photos of the food being eaten by the user through a camera and determine that the food belongs to the food type of “cabbage-vegetable-low calorie” after analyzing the food image, the smart glasses automatically determine the target number of chews as 10 according to the food type of “cabbage-vegetable-low calorie”, that is, the accumulated number of chews “10” is the target value that the user should achieve for this chewing behavior.

In this way, in this embodiment, the smart glasses determine the type of food through the food image, and then determine the corresponding target number of chews according to the type of food, so that the smart glasses can automatically determine the corresponding target number of chews for different foods eaten by the user in an intelligent manner, thereby avoiding the cumbersome operation of manually setting the target number of chews, and improving the user's using experience.

Furthermore, a second embodiment of the bone conduction-based eating monitoring method in this application is proposed according to the first embodiment of the bone conduction-based eating monitoring method described above.

In the second embodiment of the bone conduction-based eating monitoring method in this application, after step S100, the method may include:

Step S600, counting the vibration interval according to the vibration signal.

After obtaining the vibration signal of the skull related to the user's chewing behavior during eating, the smart glasses count the vibration interval of the skull according to the vibration signal.

After step S200 described above, the method may include:

Step S700, if the vibration interval reaches a preset duration period, resetting the number of consecutive chews.

It should be noted that in this embodiment, the preset duration period is a time length customized by the user or manufacturer, and is used to measure whether the chewing behavior of a user terminates. Herein, the preset duration period may be 2 seconds, 3 seconds, or 4 seconds, etc., and there is no specific limitation herein. When the number of consecutive chews of the user's chewing behavior does not reach the target number of chews and the chewing behavior terminates, a first prompt message is sent to remind the user that swallowing occurs before the user's number of consecutive chews reaches the target number of chews.

If the smart glasses monitored that the vibration interval of the skull related to the user's chewing behavior reaches the preset duration period, the smart glasses determine that the user's chewing behavior has been terminated and reset the number of consecutive chews.

Specifically, as shown in FIG. 3, assuming that the user sets the preset duration period to 2 seconds, if the smart glasses detect a vibration signal of the user's temporal bone but fail to detect the next vibration signal within 2 seconds, the smart glasses determine that the user's chewing behavior has been terminated and reset the number of consecutive chews to 0.

the method may include: Step S800, counting the number of chews according to real-time vibration signal and updating the number of consecutive chews.

When the smart glasses determine that the user's chewing behavior has been terminated and reset the number of consecutive chews and then detect the vibration signal of the skull related to the user's chewing behavior again, the smart glasses count the vibration number of the skull according to the real-time vibration signal, obtain the number of chews according to the vibration number, and update the number of consecutive chews.

Specifically, for example, assuming that the number of consecutive chews reaches 15 and the smart glasses fail to detect the 16th vibration signal of the temporal bone within 2 seconds, the smart glasses determine that the user's chewing behavior has been terminated and reset the number of consecutive chews from 15 to 0; If the smart glasses detect the vibration signal of the temporal bone again, the smart glasses recount the vibration number of the temporal bone according to the vibration signal, at this point, the monitored number of consecutive chews changes from 0 to 1 and is accumulated according to the vibration signal.

In this embodiment, after obtaining the vibration signal of the skull related to the user's chewing behavior during eating by the smart glasses, the smart glasses count the vibration interval of the skull according to the vibration signal; if the smart glasses monitored that the vibration interval of the skull related to the user's chewing behavior reaches the preset duration period, the smart glasses determine that the user's chewing behavior has been terminated and reset the number of consecutive chews; when the smart glasses determine that the user's chewing behavior has been terminated and reset the number of consecutive chews and then detect the vibration signal of the skull related to the user's chewing behavior again, the smart glasses count the vibration number of the skull according to the real-time vibration signal, obtain the number of chews according to the vibration number, and update the number of consecutive chews.

The present embodiment avoids the occurrence of incorrect accumulation for the number of consecutive chews by the smart glasses when the user eats different types of food during different monitoring periods, which affects the accuracy of monitoring results. Meanwhile, since the preset duration period can be customized by the user, it can meet the personalized needs and improve the user's using experience.

Furthermore, a third embodiment of the bone conduction-based eating monitoring method in this application is proposed according to the first embodiment of the bone conduction-based eating monitoring method described above.

In the third embodiment of the bone conduction-based eating monitoring method in this application, before the step S100, the method further includes:

Step S900, obtaining a monitoring instruction.

It should be noted that, in this embodiment, the monitoring instruction is used to trigger the smart glasses to activate the eating monitoring function. The triggering method of the monitoring instruction includes automatic triggering by the smart glasses and autonomous triggering by the user; among them, the method in which the smart glasses automatically trigger the monitoring instruction includes timer-based automatic triggering of the monitoring instruction, and the method in which the user autonomously triggers the monitoring instruction includes inputting monitoring instruction by user through voice input, or inputting monitoring instruction by user by means of touching the shortcut keys on the surface of smart glasses, and there is no specific restriction herein.

The smart glasses obtain the monitoring instruction for triggering the eating monitoring function by means of automatic triggering or user autonomous triggering.

The method further includes: Step S910, activating the eating monitoring function according to the monitoring instruction.

After receiving the monitoring instruction, the smart glasses trigger the eating monitoring function according to the monitoring instruction, to monitor the vibration signals of the skull related to the user's chewing behavior.

Specifically, for example, a user has a fixed eating schedule for three meals a day, with breakfast from 7:00 to 7:15, lunch from 12:00 to 12:30, and dinner from 18:00 to 18:30, and the user can pre-set the smart glasses to activate monitoring functions from 7:00 to 7:15, 12:00 to 12:30, and 6:00 to 6:30; when the time reaches 7:00, which belongs to the breakfast period, the smart glasses automatically trigger the monitoring instruction for triggering the eating monitoring function, and at this time, the smart glasses enter the monitoring mode, activate the eating monitoring function, and monitor the vibration signal of the user's temporal bone; alternatively, when the time reaches 12:00, which belongs to the lunch period, the smart glasses automatically trigger the monitoring instruction for triggering the eating monitoring function, and at this time, the smart glasses enter the monitoring mode, activate the food monitoring function, and monitor the vibration signal of the user's temporal bone; when the time reaches 18:00, which belongs to the dinner time, the smart glasses automatically trigger the monitoring instruction for triggering the eating monitoring function, and at this time, the smart glasses enter the monitoring mode, activate the eating monitoring function, and monitor the vibration signal of the user's temporal bone.

Specifically, for another example, when the user autonomously touches the shortcut key on the surface of the smart glasses during the eating stage, or triggers the monitoring instruction for triggering the eating monitoring function through voice input such as “activate the eating monitoring function”, the smart glasses enter the monitoring mode, activate the eating monitoring function, and monitor the vibration signal of the user's temporal bone.

In this embodiment, the monitoring instruction for triggering the monitoring function is obtained by the smart glasses by means of automatic triggering or user's autonomous triggering; after obtaining the monitoring instruction, the smart glasses trigger the eating monitoring function according to the monitoring instruction to monitor the vibration signal of skull related to the user's chewing behavior.

The present embodiment avoids the occurrence of false triggering of the eating monitoring function of the smart glasses during non-eating stages, such as when the user is chewing gum, which causes unnecessary interference to users, and reduces the power consumption of smart glasses.

In addition, referring to FIG. 6, an embodiment of the present application also proposes a bone conduction-based eating monitoring apparatus, which includes:

a monitoring module for monitoring the chewing behavior of food being eaten and obtaining vibration signal of the skull related to the chewing behavior;

a counting module for counting the number of chews according to the vibration signal to obtain the number of consecutive chews;
a prompting module for sending a first prompt information if the number of consecutive chews does not reach a target number of chews and the chewing behavior of a user terminates, and sending a second prompt information if the number of consecutive chews reaches the target number of chews.

Preferably, the bone conduction-based eating monitoring apparatus further includes:
a determining module for determining whether the number of consecutive chews reaches the preset monitoring value, and determining whether the vibration interval reaches a preset duration period; and
an obtaining module for obtaining the target number of chews if the number of consecutive chews reaches the preset monitoring value.

Preferably, the bone conduction-based eating monitoring apparatus further includes:
a reminding module for activating the eating monitoring function of the wearable device and sending a prompt message to the user if the number of consecutive chews reaches the preset monitoring value.

Preferably, the bone conduction-based eating monitoring apparatus further includes:
a determining module for obtaining a preset instruction and determining the target number of chews according to the preset instruction and for obtaining the type of food being eaten and determining the target number of chews according to the type of food.

Preferably, the counting module includes:
a counting unit for counting a vibration interval according to the vibration signal;
a resetting unit for resetting the number of consecutive chews if the vibration interval reaches a preset duration period and counting the number of chews according to real-time vibration signal and updating the number of consecutive chews.

Preferably, the monitoring module further includes:
a monitoring unit used for obtaining a monitoring instruction, and activating an eating monitoring function according to the monitoring instruction.

In addition, an embodiment of the present application further proposes a terminal device, which includes a memory, a processor, and a bone conduction-based eating monitoring program stored on the memory and operable on the processor. The steps of the bone conduction-based eating monitoring method as described above are implemented when the bone conduction-based eating monitoring program is executed by the processor.

Here, the steps implemented when the bone conduction-based eating monitoring program operated on the processor is executed may refer to various embodiments of the bone conduction-based eating monitoring method in the present application, and will not be repeated herein.

In addition, an embodiment of the present application further proposes a storage medium applied to a computer. The storage medium may be a non-volatile computer-readable storage medium, on which a bone conduction-based eating monitoring program is stored. The steps of the bone conduction-based eating monitoring method described above are implemented when the bone conduction-based eating monitoring program is executed by the processor.

Here, the steps implemented when the bone conduction-based eating monitoring program operated on the processor is executed may refer to various embodiments of the bone conduction-based eating monitoring method in the present application, and will not be repeated herein.

In addition, an embodiment of the present application further proposes a computer program product, which includes a bone conduction-based eating monitoring program. The steps of the bone conduction-based eating monitoring method as described above are implemented when the bone conduction-based eating monitoring program is executed by the processor.

Here, the steps implemented when the bone conduction-based eating monitoring program operated on the processor is executed may refer to various embodiments of the bone conduction-based eating monitoring method in the present application, and will not be repeated herein.

It should be noted that, in the descriptions, the terms “including”, “comprising”, or any other variation thereof are intended to encompass non-exclusive inclusion, such that a process, method, object, or system that includes a series of elements not only includes those elements, but also other elements that are not explicitly listed, or also include elements inherent to such a process, method, object, or system. Without further limitations, the element limited by the statement “including a . . . ” does not exclude the existence of another identical element in the process, method, object, or system that includes that element.

The above embodiments are only for description and do not represent the advantages or disadvantages of the embodiments.

Through the description of the above embodiments, those skilled in the art can clearly understand that the above method in the embodiments can be achieved through software and necessary general hardware platforms, and of course, also can be achieved through hardware. However, in many cases, the former is the better embodiments. Based on this understanding, the technical solutions of the present application, which essentially or in other words, contribute to the prior art, can be reflected in the form of a software product. The computer software product is stored in a storage medium (such as ROM/RAM, magnetic disc, optical disc), and includes several instructions to enable an intelligent express cabinet to perform the methods described in various embodiments of the present application.

The above are only preferred embodiments of the present application and are not intended to limit the patent scope of the present application. Any equivalent structure or equivalent process changes made using the description and drawings of the present application, or directly or indirectly applied in other related technical fields, are equally included in the scope of patent protection of this application.

1. A bone conduction-based eating monitoring method, wherein the bone conduction-based eating monitoring method is applied to a wearable device, and wherein the bone conduction-based eating monitoring method comprises:
monitoring a chewing behavior for food being eaten to obtain a vibration signal of a skull related to the chewing behavior;
counting a number of chews according to the vibration signal to obtain a number of consecutive chews; and
if the number of consecutive chews does not reach a target number of chews and the chewing behavior of a user terminates, sending a first prompt information; and if the number of consecutive chews reaches the target number of chews, sending a second prompt information.

2. The bone conduction-based eating monitoring method of claim 1, wherein after counting the number of chews according to the vibration signal to obtain the number of consecutive chews, the bone conduction-based eating monitoring method comprises:
determining whether the number of consecutive chews reaches a preset monitoring value; and
if the number of consecutive chews reaches the preset monitoring value, obtaining the target number of chews.

3. The bone conduction-based eating monitoring method of claim 2, wherein after determining whether the number of consecutive chews reaches the preset monitoring value, the bone conduction-based eating monitoring method further comprises:
if the number of consecutive chews reaches the preset monitoring value, activating an eating monitoring function of the wearable device and sending a prompt message to the user.

4. The bone conduction-based eating monitoring method of claim 2, wherein before obtaining the target number of chews, the bone conduction-based eating monitoring method further comprises:
obtaining a preset instruction; and
determining the target number of chews according to the preset instruction.

5. The bone conduction-based eating monitoring method of claim 2, wherein before obtaining the target number of chews, the bone conduction-based eating monitoring method further comprises:
obtaining a type of food being eaten; and
determining the target number of chews according to the type of food.

6. The bone conduction-based eating monitoring method of claim 1, wherein after obtaining the vibration signal of the skull related to the chewing behavior, the bone conduction-based eating monitoring method further comprises:
counting a vibration interval according to the vibration signal; and
wherein after counting the number of chews according to the vibration signal to obtain the number of consecutive chews, the bone conduction-based eating monitoring method further comprises:
if the vibration interval reaches a preset duration period, resetting the number of of consecutive chews; and
counting the number of chews according to a real-time vibration signal and updating the number of consecutive chews.

7. The bone conduction-based eating monitoring method of claim 1, wherein before monitoring the chewing behavior for food being eaten to obtain the vibration signal of the skull related to the chewing behavior, the bone conduction-based eating monitoring method further comprises:
obtaining a monitoring instruction; and
activating an eating monitoring function according to the monitoring instruction.

8. A bone conduction-based eating monitoring apparatus, wherein the bone conduction-based eating monitoring apparatus is applied to a wearable device, and wherein the bone conduction-based eating monitoring apparatus comprises:
a monitoring module for monitoring a chewing behavior for food being eaten to obtain a vibration signal of a skull related to the chewing behavior;
a counting module for counting a number of chews according to the vibration signal to obtain a number of consecutive chews; and
a prompting module for sending a first prompt message if the number of consecutive chews does not reach the target number of chews and the chewing behavior of a user terminates, and sending a second prompt message if the number of consecutive chews reaches the target number of chews.

9. A terminal device, wherein the terminal device comprises a memory, a processor, and a bone conduction-based eating monitoring program stored on the memory and operable on the processor, wherein the bone conduction-based eating monitoring program, when executed by the processor, implements steps of the bone conduction-based eating monitoring method according to claim 1.

10. A non-transitory storage medium on which a computer program is stored, wherein the computer program, when executed by a processor, implements steps of the bone conduction-based eating monitoring method according to claim 1.
